(19) United States
(12) Patent Application Publication
(10) Pub. No.: US 2013/0226069 A1
(43) Pub. Date: Aug. 29, 2013

(54) ACNE THERAPEUTIC AGENT AND SEBUM SECERNENT INHIBITOR WHICH COMPRISE INDOLE-3-ALKYLCARBO XYLICACID, AND KITS FOR PHOTODYNAMIC THERAPY CONTAINING THE SAME

(71) Applicant: WELSKIN CO., LTD., (US)

(72) Inventors: Dong-Seok KIM, Seoul (KR); So-Young KIM, Seoul (KR); Sun-Bang KWON, Daejon (KR)

(73) Assignee: WELSKIN CO., LTD., Seoul (KR)

(21) Appl. No.: 13/726,428

(22) Filed: Dec. 24, 2012

Related U.S. Application Data
(62) Division of application No. 12/452,348, filed on Apr. 30, 2010, now abandoned.

(30) Foreign Application Priority Data
Jun. 29, 2007 (KR) 10-2007-0065046

(51) Int. Cl. A61K 41/00

(52) U.S. Cl. CPC A61K 41/0057
USPC 604/20

(57) ABSTRACT

The present invention relates to novel uses of indole-3-alkylcarboxylic acid and derivatives thereof as a photosensitizer for the treatment of acne or for the inhibition of sebum secretion. More particularly, the present invention is directed to a photosensitizer for photodynamic therapy, a photodynamic therapy kit and photodynamic therapeutic composition comprising thereof, which contain indole-3-alkylcarboxylic acid being activated by light. The light which activates indole-3-alkylcarboxylic acid of the present invention may be ultraviolet rays or visible lights, preferably blue light or green light.

### TECHNICAL FIELD

The present invention relates to novel uses of indole-3-alkylcarboxylic acid and derivatives thereof as a photosensitizer for the treatment of acne or for the inhibition of sebum secretion. More particularly, the present invention is directed to a photosensitizer for photodynamic therapy, a photodynamic therapy kit and photodynamic therapeutic composition comprising thereof, which contain indole-3-alkylcarboxylic acid being activated by light. The light which activates indole-3-alkylcarboxylic acid of the present invention may be ultraviolet rays or visible lights, preferably blue light or green light.

### BACKGROUND ART

Photodynamic therapy (PDT) is one of the new promising therapies for the treatment of cancer. In the photodynamic therapy, a cancer is treated in such a way that singlet oxygens or free radicals which created by the reaction of a photosensitizer with oxygen and light.

There are several advantages of photodynamic therapy in the treatment of cancer. First of all, selective destruction of cancer cells are possible without damaging normal cells. Owing to this advantage, local anesthesia is usually enough for the treatment. No requirement for general anesthesia enabled rapid recovery of patients and can reduce socioeconomic cost for the treatment of cancer.

Photodynamic therapy research began in 1980s, and was approved for clinical surgery operations in Canada, Germany, Japan, etc. in 1990s. The first PDT application, which was approved by the U.S. FDA in January 1996, was the palliative treatment of obstructive esophageal cancer. Then, in September 1997, FDA approved the first treatment of lung cancer using PDT. According to statistical data of early 1996, there were about 3,000 photodynamic therapies in about 32 nations.

However, the presently operated PDT is limited because the light is unable to penetrate deeply when treating large tumors. In addition, photosensitizers such as porphyrin is expensive, and there are risks for side effects such as phototoxicity due to their slow metabolism. Furthermore, the concentration of the photosensitizer within tumors are very low. Therefore, it is difficult to treat cancers by PDT.

Photofrin™, standard photosensitizer approved by FDA in 1996, is known to have reasonable therapeutic effects and stability. However, this drug is known to be accumulated for 5 to 6 weeks after single administration, and therefore may cause side effects. Furthermore, synthesis of highly pure Photofrin™ is difficult and, although light of wavelength of 650 nm to 850 nm is optimal for PDT, Photofrin™ absorbs light of wavelength of about 630 nm, which can penetrate tumors only a few millimeters, thereby making PDT for the treatment of cancer inefficient (Chemistry & Industry, Sep. 21, 1998, 739-743: Chemical & Engineering News, Nov. 2, 1998, 22-27). Consequently, there is still great demand for the development of an effective photosensitizer for use in PDT.

The next generation photosensitizing agents such as porphyrins, chlorins, bacteriochlorins, porphycenes, etc. are being researched extensively (J Org. Chem., 63, 1998, 1646-1656). Among these agents, much research continues to be carried out on pheophytins, which is chlorophyll with its metal ions removed. Pheophytins not only absorb light with long wavelengths better than Photofrin3, a derivative of hematoporphyn, but can also be separated and prepared with high purity. However, despite extensive research, no real substantial results have been attained yet.

Recently, PDT are also being used for the treatment of psoriasis and acne. For the treatment of acne, aminolevulinic acid (ALA) is commonly used but light exposure should be restricted for certain periods of time. In addition, ALA-PDT is painful and frequently induced hyperpigmentation as an adverse effect. Thus, a new effective and safe photosensitizer needs to be developed. Recently, the present inventors had found out that indole-3-alkylcarboxylic acid can be effective for the treatment of cancer when indole-3-alkylcarboxylic acid is combined with light (Korean Patent Application No. 10-2006-0063841). However, the present inventors did not find that this combination could be effective in the treatment of acne and also in the inhibition of sebum secretion.

The present inventors have found that indole-3-alkylcarboxylic acid may be used as an acne therapeutic agent and sebum secretion inhibitor, and have invented the present invention.

### DISCLOSURE

### Technical Problem

The primary object of the present invention is to provide a photosensitizer for the treatment of acne comprising indole-3-alkylcarboxylic acid.

Another object of the present invention is to provide a photosensitizer for the inhibition of sebum secretion comprising indole-3-alkylcarboxylic acid.

Still another object of the present invention is to provide a photodynamic therapy kit and a photosensitizing pharmaceutical composition for the treatment of acne and for the inhibition of sebum secretion, which comprise indole-3-alkylcarboxylic acid.

### Technical Solution

The above-mentioned primary object of the present invention can be accomplished by examining the pharmacological effect of indole-3-alkycarboxylic acid as a photosensitizer for the treatment of acne and for the inhibition of sebum secretion.

Another object of the present invention can be accomplished by providing a photodynamic therapy kit and a photosensitizing pharmaceutical composition for the treatment of acne and for the inhibition of sebum secretion, which comprise indole-3-alkylcarboxylic acid being activated by light.

The light which activates indole-3-alkylcarboxylic acid of the present invention may be ultraviolet rays or visible lights, preferably blue light or green light.

Indole-3-acetic acid (IAA) is a member of the group of phytohormones called auxins. Auxins, which are known to hormones regulating plant growth, stimulate cell elongation in the stem and inhibit cell elongation in the root. Due to the action of auxins, stems show positive phototropism and negative gravitropism. Indole-3-acetic acid has been known to have anti-cancer effects for long time, however action mechanism of IAA is not known well.

The present inventors have found that IAA alone is non-toxic and well tolerated in humans, however becomes active so as to necrose cancer cells after oxidative decarboxylation by horseradish peroxidase (HRP) (Kim et al., Oxidation of indole-3-acetic acid by horseradish peroxidase induces apoptosis in G361 human melanoma cells, Cell Signal 2004; 16: 81-8). However, when using HRP, HRP must be targeted specifically to cancer cells. Such targeting causes immunological problems, metabolic problems in the liver, and other technical problems. When HRP is not targeted specifically to cancer cells, even if these problems are overcome, it is difficult to apply HRP clinically. Consequently, it has limitations to use IAA for the treatment of cancer.

Since it is difficult to deliver HRP specifically to cancer cells, the present inventors have tried to find out another method which can activate IAA, and have found that light can activate IAA and similar results compared to those by HRP can be obtained. IAA can be activated by visible and ultraviolet light. Among visible light, green and blue light is especially effective in the activation of IAA. Ultraviolet light can also activate IAA and can be used in the treatment of many different diseases including cancer.

The present inventors have found that IAA can work as a photosensitizer and also can be effective in the treatment of acne and the inhibition of sebum secretion. In addition, blue and green light seemed to be most promosing light source for these purposes.

In addition, the inventors have found that IAA can be used as a photosentizer for the treatment of acne or suppression of sebum secretion, and the combination of IAA and light have bactericidal effects on P. acnes or S. aureus (FIG. 1).

Furthermore, the present inventors have found that the combination of IAA and light is very effective in the control of sebum secretion as well as the treatment of acne (FIG. 2). As yet, mechanism of sebum controlling action has been unknown, however the present invention can be used for this purpose and also for esthetic purpose.

In order to accomplish the aforementioned objects, the present invention provides a photosensitizer for the treatment of acne comprising indole-3-alkylcarboxylic acid of formula (I) or pharmaceutically acceptable salt thereof:

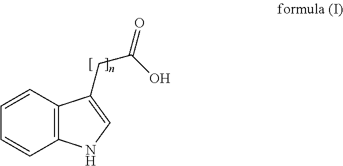

where n is an integer of 0 to 3.

Also, the present invention provides a photosensitizing pharmaceutical composition for the treatment of acne and the inhibition of sebum secretion, comprising pharmaceutically effective amount of indole-3-alkylcarboxylic acid of the formula (I) or pharmaceutically acceptable salt thereof.

The present invention also provides a photodynamic therapy kit comprising indole-3-alkylcarboxylic acid of the formula (I) or pharmaceutically acceptable salt thereof, and a light source for in vivo or in vitro irradiation of light.

Hereinafter, the present invention will be described in detail.

Indole-3-alkylcarboxylic acid of the formula (I) does not need any other photocatalysts for photosensitization, and acts in itself as a photosensitizer. Therefore, indole-3-alkylcarboxylic acid of the formula (I) is activated by light and has a bacteriocidal effect on skin bacteria such as P. acnes, S. aureus, etc. Ultraviolet or visible light can activate indole-3-alkylcarboxylic acid, regardless of its wavelength. However, when indole-3-alkylcarboxylic acid is activated in vivo, longer wavelength light (>280 nm) seemed to be safe considering harmful effect on normal tissue. Since irradiation time increases as wavelength decreases, it is preferable to use light of wavelength of 280 nm to 1,000 nm, more preferably 300 nm to 750 nm.

Considering the photoactivation efficeincy of indole-3-alkylcarboxylic acid, it is preferable to use ultraviolet ray of wavelength of 350 nm to 400 nm, blue light of wavelength of 400 nm to 500 nm, or green light of wavelength of 500 nm to 600 nm. Moreover, it is most perferable to use blue or green light, considering degree of activation, cell penetration and in vivo safety of indole-3-alkylcarboxylic acid.

The light souce for radiation of light may be at least one of an light source for the in vitro radiation selected from the group consisting of an ultrasound radiation emitter, a light emitting diode, a laser diode, a dye laser, a metal halide lamp, a flashlamp, a mechanically filtered fluorescent light source, and a mechanically filtered incandescent or filamentous light source; and a laser fiber for photodynamic treatment by the in vivo radiation. When indole-3-alkylcarboxylic acid is irradiated by light, indole-3-alkylcarboxylic acid may be activated by being exposed to light of one or more wavelengths during therapeutically effective pulse duration time. For the activation indole-3-alkylcarboxylic acid, there is no limitation on the intensity of light. If the intensity of light is weak, the duration time and/or frequency of the pulse can be increased for the activation of indole-3-alkylcarboxylic acid, and vice versa.

If the light intensity is too low, the light will not sufficiently penetration the target tissue and thus effective light activation will not occur. If the light intensity is too high, on the other hand, necrosis of normal tissue may occur. Thus, the intensity of light should be maintained between 1 J/cm² to 100 J/cm². Further, if the pulse duration time is too short or the pulse delivery frequency is too low, the effectiveness of light activation will be diminished. Also, if pulse duration time is too long or the pulse delivery frequency is too high, necrosis of normal tissue may occur. In addition, as mentioned above, the duration time and frequency of the pulse should be adjusted based on the intensity of light. That is, if the intensity of light is high, the duration time and frequency of the pulse should be decreased. Therefore, the duration time and frequency of the pulse may be adjusted based on the intensity of light, the side effects to normal tissues, etc. Although the duration time and frequency of the pulse cannot be determined uniformly, the pulse duration time may be preferably maintained between 0.1 ms to 500 ms and the radiation number of the pulse may be preferably maintained between 1 to 100. For the treatment of acne, according to an embodiment of the present invention, the intensity of light is 20 J/cm², the pulse duration time is 15 ms, and the radiation number of the pulse is 1. Preferably, intense pulse lihgt may be used for the activation of indole-3-alkylcarboxylic acid.

The photosensitizing pharmaceutical composition of the present invention comprises 0.001 wt % to 99 wt % of indole-3-alkylcarboxylic acid, preferably 0.001 wt % to 30 wt % of indole-3-alkylcarboxylic acid. In order to maintain sufficient the photosensitivity effect and therapeutic effect of indole-3-alkylcarboxylic acid, the weight of indole-3-alkylcarboxylic acid should be at least 0.001 wt %. The formulation of the composition may be in the form selected from liquid, semisolid, solid or aerosol. For example, the formulation of the composition may be in the form selected from aqueous or non-aqueous suspension, solution, cream, ointment, gel, syrup, suppository, tablet, capsule or micro-droplet spray.

In addition, the composition may further comprise excipients in order for the formulation. Also, the composition may comprise at least on selected from preservatives, stabilizers, buffers, pH regulators, sweetening agents, aromatic agents, coloring agents, etc., for storage and administration thereof. In addition, other types of drugs may be added to the composition based on the objective of the therapy.

Furthermore, indole-3-alkylcarboxylic acid may be photoactivated either after applying to the body or before applying to the body.

The photosensitizer or photodynamic therapy kit of the present invention is effective for the treatment of acne or inhibition of sebum secretion.

### Advantageous Effects

Photoactivated indole-3-aklylcarboxylic acid and derivatives thereof according to the present invention can show bacteriocidal effect and also control sebum secretion. Consequently, the present invention provides a novel use of indole-3-acetic acid and derivatives thereof as a photosensitizer for the treatment of acne or suppression of sebum secretion. Visble and ultraviolet light can activate indole-3-aklylcarboxylic acid, and green and blue light is preferred.

### DESCRIPTION OF DRAWINGS

FIG. 1 shows the bactericidal effects of light and indole-3-acetic acid on Propionibacterium acnes in Example 1.

FIG. 2 shows the bactericidal effects of light and indole-3-acetic acid on Staphylococcus aureus in Example 2.

FIG. 3 shows the therapeutic effects of light and indole-3-acetic acid on acne in Example 3.

FIG. 4 shows the inhibitory effects of light and indole-3-acetic acid on sebum secretion in Example 4.

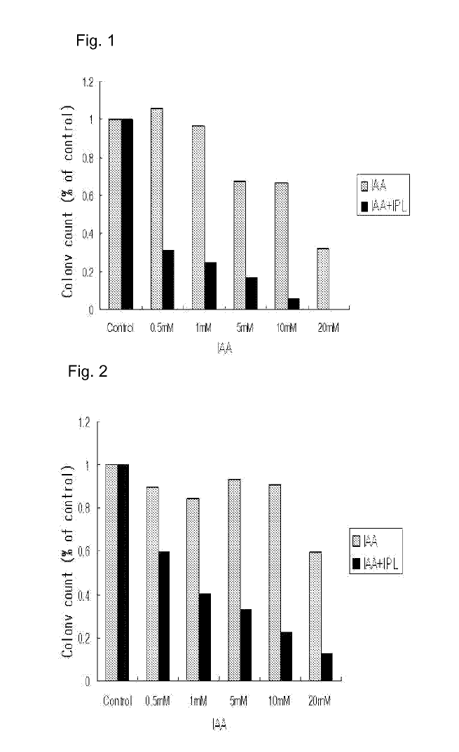

### BEST MODE

Hereinafter, the present invention will be described in greater detail with reference to the following examples. The examples are given only for illustration of the present invention and not to be limiting the scope of the present invention.

### EXAMPLE 1

### Bacteriocidal Effects on Propionibacterium acnes and Skin Bacteria by the Combination of indole-3-acetic Acid and Light

In this experiment, the present inventors found that bacteriocidal effects of indole-3-acetic acid (IAA) is shown only when light is simultaneously irradiated, and IAA alone is not toxic to bacteria. P. acnes 706486 and S. aureus W-1-14 was used for this experiment. Mueller Hinton II broth (Becton, Dickinson Co., Sparks, U.S.A) was used for culture of bacteria. The cultured S. aureus colony was dissolved with phosphate buffered saline (PBS, Invitrogen Co., NY, U.S.A) such that the McFarland turbidity became 0.5. The resultant bacterial solution was at the concentration of 2×10⁴/ml. Then, the bacterial solution was further diluted to the concentration of 1×10⁸/ml and 10 μl of the bacterial solution was spread onto the Mueller Hinton II agar (Becton, Dickinson Co., Sparks, U.S.A) plate followed by drying in clean bench for 30 min. IAA was dissolved with Dulbecco's phosphate buffered saline (DPBS, Invitrogen Co., NY, U.S.A) to obtain samples of IAA concentration of 20 mM, 10 mM, 5 mM, 1 mM and 0.5 mM. Three minutes after adding 4 ml of 0 mM, 0.5 mM, 1 mM, 5 mM, 10 mM, and 20 mM IAA solution to 6 bacterial solution inoculated mediums respectively, Intermittent Pulse Laser (IPL, Ellipse Flex DDD, Denmark) was irradiated to the mediums. 10 J/cm² of the IPL of wavelength of 400 nm to 720 nm was irradiated to the mediums, using an applicator equipped with a filter. One minute after IPL irradiation, the mediums were washed for two times with PBS and incubated at 37° C. for 24 hrs and then the number of colonies was counted respectively. Four minutes after adding 4 ml of 0 mM, 0.5 mM, 1 mM, 5 mM, 10 mM, and 20 mM IAA solution to another 6 bacterial solution inoculated mediums, the mediums were treated as the above-mentioned process except that IPL was not irradiated, and then the number of colonies was counted respectively. The above-mentioned process was repeated except that P. acnes inoculated mediums were employed in place of S. aureus inoculated mediums and cultured in an anaerobic chamber substituted with nitrogen, and then the number of colonies was counted.

The number of colonies of P. acnes and S. aureus treated with IAA and IPL was compared with that of P. acnes and S. aureus treated with only IAA. IAA with IPL irradiation showed dramatic inhibitory effects on P. acnes proliferation. These bacteriocidal effect was definitely observed from 0.5 mM to 20 mM IAA solutions, and there was no growth of bacteria when using 20 mM IAA solution. When 5 WA or more IAA solution was added, the number of colonies was decreased to 68% of control group, even though IPL was not irradiated. Therefore, it seems that 5 mM or more IAA solution inhibits the growth of P. acnes.

Same results were obtained for the S. aureus. IPL irradiation and 0.5 mM IAA solution reduced the number of colonies to 63% of control group. IPL irradiation and 1 mM IAA solution more significantly reduced the number of colonies to 32% of control group. As the concentration of IAA solution increased, the number of colonies decreased after IPL irradiation. Groups without IPL irradiation showed that the number of colonies was similar to that of control group after treating with IAA. However, when treated with 20 WA IAA solution, the number of colonies decreased to 60% of control group. The growth of S. aureus was also inhibited by IAA as well as P. acnes when the concentration of IAA which was not photoactivated was sufficiently high.

### EXAMPLE 2

### Improvement of Acne by the Combination of IAA and Light

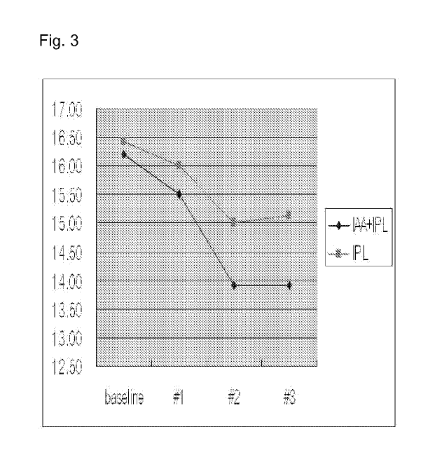

IAA was applied to only half of face and 20 J of IPL was irradiated. The face was irradiated 3 times at 2 weeks intervals and the number of inflammatory lesions was counted. As a result, case-IPL only group did not showed any statistically significant differences, however case-IAA with IPL group showed significant improvement (FIG. 3).

TABLE 1
Number of inflammatory lesion after 2, 4, and 6 weeks with the
treatment with IAA and light (n = 14)

| | IAA + IPL | | IAA + IPL | | IAA + IPL |
|---|---|---|---|---|---|
| | (mean ± SD) | | (mean ± SD) | | (mean ± SD) |
| T0 | 16.21 ± 9.18 | T0 | 16.21 ± 9.18 | T0 | 16.21 ± 9.18 |
| T1 | 15.50 ± 9.03 | T2 | 13.93 ± 8.54 | T3 | 13.93 ± 8.30 |
| T1-T0 | −0.71 ± 2.20 | T2-T0 | −2.29 ± 3.36 | T3-T0 | −2.29 ± 3.41 |
| p | 0.246 | p | 0.024 | p | 0.026 |
| value | | value | | value | |

* Probability p (paired t-test, statistical significance: p < 0.05)

TABLE 2
Number of inflammatory lesion after 2, 4, and 6 weeks with
the treatment with light only (n = 14)

| | IPL | | IPL | | IPL |
|---|---|---|---|---|---|
| | (mean ± SD) | | (mean ± SD) | | (mean ± SD) |
| T0 | 16.43 ± 11.49 | T0 | 16.43 ± 11.49 | T0 | 16.43 ± 11.49 |
| T1 | 16.00 ± 11.02 | T2 | 15.00 ± 10.85 | T3 | 15.14 ± 11.33 |
| T1-T0 | −0.43 ± 1.87 | T2-T0 | −1.43 ± 2.87 | T3-T0 | −1.29 ± 3.05 |
| p | 0.407 | p | 0.086 | p | 0.139 |
| value | | value | | value | |

* Probability p (paired t-test, statistical significance: p < 0.05)

### EXAMPLE 3

Sebum Secretion Inhibitory Effects by the Combination of IAA and IPL

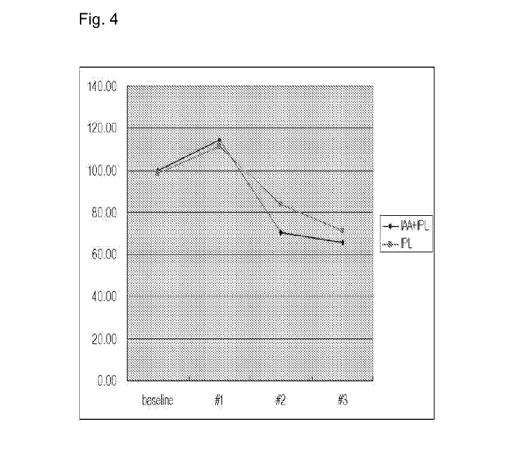

As described in Example 2, IAA was applied to only half of face and 20 J of IPL was irradiated. The face was irradiated 3 times at 2 weeks intervals and the amount of sebum secretion was measured. As a result, case-IPL only group did not showed any statistically significant differences, however case-IAA with IPL group showed significant inhibitory effect of sebum secretion (FIG. 4).

TABLE 3
Changes of sebum secretion after IAA and light treatment
after 2, 4, and 6 weeks (n = 14)

| | IAA + IPL | | IAA + IPL | | IAA + IPL |
|---|---|---|---|---|---|
| | (mean ± SD) | | (mean ± SD) | | (mean ± SD) |
| T0 | 99.64 ± 56.93 | T0 | 99.64 ± 56.93 | T0 | 99.64 ± 56.93 |
| T1 | 114.71 ± 63.83 | T2 | 70.43 ± 42.80 | T3 | 65.79 ± 27.90 |
| T1-T0 | 15.07 ± 69.70 | T2-T0 | −29.21 ± 45.67 | T3-T0 | −33.86 ± 44.98 |
| p | 0.433 | p | 0.032 | p | 0.015 |
| value | | value | | value | |

* Probability p (paired t-test, statistical significance: p < 0.05)

TABLE 4
Changes of sebum secretion after light only treatment after
2, 4, and 6 weeks (n = 14)

| | IPL | | IPL | | IPL |
|---|---|---|---|---|---|
| | (mean ± SD) | | (mean ± SD) | | (mean ± SD) |
| T0 | 98.50 ± 76.40 | T0 | 98.50 ± 76.40 | T0 | 98.50 ± 76.40 |
| T1 | 111.43 ± 81.20 | T2 | 83.86 ± 69.19 | T3 | 71.14 ± 44.92 |
| T1-T0 | 12.93 ± 67.74 | T2-T0 | −14.64 ± 33.28 | T3-T0 | −27.36 ± 49.94 |
| p | 0.488 | p | 0.124 | p | 0.061 |
| value | | value | | value | |

* Probability p (paired t-test, statistical significance: p < 0.05)

1-24. (canceled)

25. A photodynamic acne therapy kit comprising:
i) a photosensitizing pharmaceutical composition for the treatment of acne comprising 0.001 wt % to 30 wt % of indole-3-alkylcarboxylic acid of formula (I) or pharmaceutically acceptable salt thereof; and 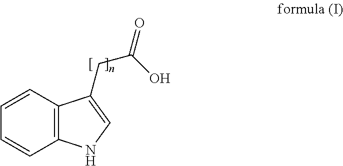
ii) a light source for irradiation of a light of wavelength of 280 nm to 1,000 nm
where n is an integer of 0 to 3.

26. The photodynamic acne therapy kit of claim 25, wherein said light source irradiates ultraviolet ray of wavelength of 350 nm to 450 nm, blue light of wavelength of 400 nm 500 nm, or green light of wavelength of 500 nm 600 nm.

27. The photodynamic acne therapy kit of claim 25, wherein said light source is at least one of a light source for the in vitro irradiation selected from the group consisting of an ultrasound radiation emitter, a light emitting diode, a laser diode, a dye laser, a metal halide lamp, a flashlamp, a mechanically filtered fluorescent light source, and a mechanically filtered incandescent or filamentous light source; and a laser fiber for photodynamic treatment by the in vivo radiation.

28. The photodynamic acne therapy kit of claim 26, wherein an intensity of a light irradiated by said light source is 1 J/cm² to 100 J/cm².

29. The photodynamic acne therapy kit of claim 28, wherein a pulse duration time of the light irradiated by said light source is between 0.1 ms and 500 ms, and the number of irradiation is between 1 and 100.
